(19) United States
(12) Patent Application Publication
(10) Pub. No.: US 2016/0168537 A1
(43) Pub. Date: Jun. 16, 2016

(54) DIFFERENTIATED CELLULAR COMPOSITIONS AND METHODS FOR THEIR PREPARATION AND USE

(71) Applicant: LIFE TECHNOLOGIES CORPORATION, Carlsbad, CA (US)

(72) Inventors: Jonathan JACKSON, Cary, NC (US); Stephen FERGUSON, Apex, NC (US); Rafal WITEK, Burlington, NC (US)

(21) Appl. No.: 14/967,085

(22) Filed: Dec. 11, 2015

Related U.S. Application Data
(63) Continuation of application No. 13/829,645, filed on Mar. 14, 2013, now abandoned.

(51) Int. Cl. C12N 5/071

(52) U.S. Cl. CPC C12N 5/067; C12N 2506/14; C12N 2533/90

(57) ABSTRACT

The present invention relates to methods for culturing progenitor cells to differentiate primarily into a single population of cells with the same phenotype, and to compositions thereof. In particular, it relates to methods for culturing liver progenitor cells to differentiate primarily into a single population of hepatocyte-like cells, and to compositions thereof.

### TECHNICAL FIELD

The present invention relates to methods for culturing progenitor cells to differentiate primarily into a single population of cells with the same phenotype, and to compositions thereof. In particular, it relates to methods for culturing liver progenitor cells to differentiate primarily into a single population of hepatocyte-like cells, and to compositions thereof.

### BACKGROUND

Currently, primary human hepatocytes are used as the gold standard in vitro hepatic model system due to their ability to support mature hepatic phenotypes, such as metabolism, transport, and induction, which are important in drug clearance, drug-drug interaction, and safety assessments. However, limited availability, lot-to-lot variability, short life span, finite lot sizes and cost have limited the use of primary human hepatocytes for drug screening applications to identify potential drug-drug interaction and safety liabilities.

Cell lines, such as HepG2 and Fa2N-4, have been tried as alternative model systems for certain screening applications but have failed to support the broader complement of primary human hepatocyte functionality, such as basal metabolism, biliary polarization, or lack of regulatory factors. There remains a need for readily-available, reproducible cell cultures which support mature hepatic phenotypes for xenobiotic and other studies but without the limitations of primary hepatocytes.

All patents, patent applications, and publications cited herein are hereby incorporated herein by reference in their entirety.

### SUMMARY

Provided herein, in part, are improved methods for preparing a progenitor cell-derived cell preparation substantially comprised of hepatocyte-like cells. Also provided are compositions comprising substantially comprised hepatocyte-like cells.

In one aspect, provided herein is a method for making a preparation substantially comprised of hepatocyte-like cells, the method comprising inducing differentiation of hepatic progenitor cells and culturing the cells with an overlay comprising at least one extracellular matrix protein or fragment thereof. In some embodiments, the hepatic progenitor cells are induced to differentiate before culturing with the overlay. In other embodiments, the hepatic progenitor cells are induced to differentiate while culturing with the overlay. In some embodiments, the preparation comprises >70% hepatocyte-like cells.

In another aspect, provided herein is an in vitro cell culture comprising a population of differentiated cells derived from a hepatic progenitor cell line where the population of differentiated cells comprises >70% hepatocyte-like cells. In some embodiments, the population of differentiated cells comprises >80% hepatocyte-like cells. In other embodiments, the population of differentiated cells comprises >90% hepatocyte-like cells.

In another aspect, provided herein is a composition comprising an in vitro population of differentiated cells derived from a hepatic progenitor cell line and a matrix overlay, where the overlay comprises at least one extracellular matrix protein or fragment thereof In some embodiments, the matrix overlay comprises at least three extracellular matrix proteins or fragments thereof. In another embodiment, the matrix overlay also comprises an agent capable of inducing differentiation of the progenitor cell line. In some embodiments, the population of differentiated cells in the composition comprises >70% hepatocyte-like cells. In other embodiments, the population of differentiated cells in the composition comprises >80% and >90% hepatocyte-like cells.

In another aspect, provided herein is a method for increasing cytochrome P450 activity in an in vitro cell preparation, the method comprising inducing differentiation of hepatic progenitor cells, culturing the cells with a matrix overlay for at least one day, where the overlay comprises at least one extracellular matrix protein or fragment thereof, and measuring cytochrome P450 activity in the cell preparation, whereby the activity in the cell preparation is increased relative to a culture of the progenitor cells differentiated and cultured without a matrix overlay.

In some embodiments of the provided methods and compositions the hepatic progenitor cells are HepaRG™ cells.

In another aspect, provided herein is a differentiated population of HepaRG™ cells comprising >70% hepatocyte-like cells. In some embodiments, the differentiated population of HepaRG™ cells comprises >80% hepatocyte-like cells. In other embodiments, the differentiated population of HepaRG™ cells comprises >90% hepatocyte-like cells.

### BRIEF DESCRIPTION OF THE DRAWINGS

FIG. 1A and FIG. 1B depict phase-contrast microscopy images of HepaRG cells having been cultured without (FIG. 1A) and with (FIG. 1B) a matrix overlay in serum-free medium (750).

FIG. 2A and FIG. 2B depict phase-contrast microscopy images of HepaRG cells having been cultured without (FIG. 2A) and with (FIG. 2B) a matrix overlay in medium with serum (740).

FIG. 3A and FIG. 3B depicts images of HepaRG cells having been cultured without (FIG. 3A) and with (FIG. 3B) a matrix overlay in plating medium (770). Immunohistochemistry was used to detect CYP3A4 in the cell cultures.

FIG. 4 is a chart depicting the induction of CYP1A2 activity in HepaRG cells treated with DMSO with or without a matrix overlay and in the presence of serum-containing (740) or serum-free (750) media.

FIG. 5 is a chart depicting the induction of CYP1A2 activity in HepaRG cells cultured with or without a matrix overlay and in the presence of serum-containing (740) or serum-free (750) media. The cells were treated with DMSO, omeprazole (OMP), Phenobarbital (PB), or Rifampicin (RIF).

FIG. 6 is a chart depicting the induction of CYP2B6 activity in HepaRG cells treated with DMSO with or without a matrix overlay and in the presence of serum-containing (740) or serum-free (750) media.

FIG. 7 is a chart depicting the induction of CYP2B6 activity in HepaRG cells cultured with or without a matrix overlay and in the presence of serum-containing (740) or serum-free (750) media. The cells were treated with DMSO, OMP, PB, or RIF.

FIG. 8 is a chart depicting the induction of CYP3A4 activity in HepaRG cells treated with DMSO with or without a matrix overlay and in the presence of serum-containing (740) or serum-free (750) media.

FIG. 9 is a chart depicting the induction of CYP3A4 activity in HepaRG cells cultured with or without a matrix overlay and in the presence of serum-containing (740) or serum-free (750) media. The cells were treated with DMSO, OMP, PB, or RIF.

FIG. 10 is a chart depicting the induction of CYP1A2 mRNA in HepaRG cells treated with DMSO with or without a matrix overlay and in the presence of serum-containing (740) or serum-free (750) media.

FIG. 11 is a chart depicting the induction of CYP1A2 mRNA in HepaRG cells cultured with or without a matrix overlay and in the presence of serum-containing (740) or serum-free (750) media. The cells were treated with DMSO, OMP, PB, or RIF.

FIG. 12 is a chart depicting the induction of CYP2B6 mRNA in HepaRG cells treated with DMSO with or without a matrix overlay and in the presence of serum-containing (740) or serum-free (750) media.

FIG. 13 is a chart depicting the induction of CYP2B6 mRNA in HepaRG cells cultured with or without a matrix overlay and in the presence of serum-containing (740) or serum-free (750) media. The cells were treated with DMSO, OMP, PB, or RIF.

FIG. 14 is a chart depicting the induction of CYP3A4 mRNA in HepaRG cells treated with DMSO with or without a matrix overlay and in the presence of serum-containing (740) or serum-free (750) media.

FIG. 15 is a chart depicting the induction of CYP3A4 mRNA in HepaRG cells cultured with or without a matrix overlay and in the presence of serum-containing (740) or serum-free (750) media. The cells were treated with DMSO, OMP, PB, or RIF.

FIG. 16 is a chart depicting CYP1A2 activity in HepaRG cells differentiated with or without a matrix overlay (DM) and undifferentiated HepaRG cells cultured in growth medium with or without a matrix overlay (GM).

FIG. 17 is a chart depicting CYP2B6 activity in HepaRG cells differentiated with or without a matrix overlay (DM) and HepaRG cells cultured in growth medium with or without a matrix overlay (GM).

FIG. 18 is a chart depicting CYP3A4 activity in HepaRG cells differentiated with or without a matrix overlay (DM) and HepaRG cells cultured in growth medium with or without a matrix overlay (GM).

### DETAILED DESCRIPTION

We have discovered methods and compositions for culturing hepatic progenitor cells so that they differentiate primarily into a single population of cells with the same phenotype.

Hepatic progenitor cell lines can retain many characteristics of primary hepatocytes. Differentiated hepatocytes derived from such progenitor cells are well suited for use in drug screening and toxicity studies. Derivation of differentiated cells from a progenitor cell line, however, often results in a population of cells containing more types of cells than the desired cell type. A mixed cell culture can result in data complexity and inefficiency in assay performance posing challenges to drug screening, drug safety, and research studies.

The HepaRG™ cell line is a bi-potent hepatic progenitor cell line that differentiates into two distinct hepatic cell types: hepatocyte-like cells and cholangiocyte-like cells (Gripon et al. (2002) Proc. Natl. Acad. Sci. USA 99:15655-15660; Cerec et al. (2007) Hepatology 45:957-967). Terminally differentiated HepaRG™ cells have been shown to support mature hepatic phenotypes and have comparable functionality to primary human hepatocytes. The reproducibility of metabolic, transport, and induction responsiveness in HepaRG™ cells make these cells an excellent hepatic model system for drug metabolism and toxicity studies, including such as prediction of xenobiotic clearance, drug-drug interactions, and safety assessment.

HepaRG™ progenitor cells can be induced to terminally differentiate using a standard practice of culturing with 2% dimethyl sulfoxide (DMSO) upon reaching confluency which results in the formation of a population with about 50% hepatocyte-like cells and about 50% cholangiocyte-like cells. Use of a cell culture in which only about 50% of the population exhibits a hepatocyte-like phenotype can make it difficult to ascertain which cell type in the culture contributed to the overall effect observed in an assay.

Disclosed herein are methods and compositions which relate, in part, to the culturing and differentiation of hepatic progenitor cells. Use of the methods and compositions provided herein lead to a differentiated hepatic progenitor cell population primarily composed of a single population of cells with the same phenotype. In exemplary embodiments, use of the provided methods and compositions result in culture preparations which are substantially composed of hepatocyte-like cells thus enabling the use of such cell cultures in, for example, drug investigation studies.

In certain of the provided methods, undifferentiated hepatic progenitor cells are plated in a culture vessel with media that promotes differentiation and with an extracellular matrix (ECM) overlay. Media and agents which promote differentiation of hepatic progenitor cells are known in the art. The cells are maintained in differentiation media and with the overlay until the population's differentiation and/or confluency reach a desired extent. During this culture period, the media and overlay are renewed as needed. For example, the differentiation media is renewed every 2-3 days and the overlay is renewed every 2-7 days.

Culturing the cells with an ECM overlay stimulates the progenitor cells to preferentially differentiate into hepatocyte-like cells. The resultant cell population has a substantially uniform phenotype and an improved function and performance in, for example, drug screening and metabolic assays compared to the differentiated cell population cultured without an overlay.

As noted above, hepatic progenitor cells are known in the art and include, but are not limited to, HepaRG™ cells. In other embodiments, progenitor cells such as induced pluripotent stem cells (iPSCs), induced to differentiate into hepatocyte-like cells are also of use in the methods and compositions provided.

In certain embodiments, the hepatic progenitor cells are incubated in differentiation media prior to the addition of the ECM overlay. Accordingly, in some embodiments, the progenitor cells may be incubated in differentiation media at least about 8 hours prior to addition of the ECM overlay. In other embodiments, the progenitor cells may be incubated in differentiation media at least about 12 hours prior to addition of the ECM overlay. In other embodiments, the progenitor cells may be incubated in differentiation media about 24 hours or about 48 hours prior to addition of the ECM overlay.

In certain embodiments, differentiation media and the ECM overlay are introduced to the hepatic progenitor cells concurrently or at about the same time, that is, within about 4 hour period, both the differentiation media and the ECM overlay are added to the progenitor cells. In some embodiments, both the differentiation media and the ECM overlay are introduced to the progenitor cells within about a two hour period. In other embodiments, both the differentiation media and the ECM overlay are introduced to the progenitor cells within about a one hour period. In other embodiments, both the differentiation media and the ECM overlay are introduced to the progenitor cells within about a 30 minute period.

In some embodiments, hepatic progenitor cells plated in a culture vessel are overlayed with an ECM and cultured in differentiation medium, with the differentiation media and the overlay renewed every few days as needed. For example, the cells may be cultured in these conditions for 14 days with the differentiation media being renewed every 2-3 days and the overlay being renewed every 2-7 days.

In other embodiments, hepatic progenitor cells are differentiated then cryopreserved before being thawed, plated and cultured with an ECM overlay. For example, cryopreserved differentiated HepaRG™ cells are thawed and plated. After the cells have attached to the culture vessel, the media is removed, cells are overlayed with an ECM, and culturing is resumed. In embodiments when the progenitor cells are differentiated prior to cryopreservation, the cells may be thawed and cultured in a media without a differentiation agent(s). In such cases, a general purpose growth medium without differentiation agents may also be used for the ECM overlay. For example, cryopreserved differentiated HepaRG™ cells may thawed and plated in a general purpose growth medium which does not include an agent(s) for inducing differentiation of undifferentiated HepaRG™ cells. Four to thirty hours, for example, after plating the differentiated HepaRG™ cells, an ECM overlay is added to the cells and the cells continue in culture with the ECM overlay.

An extracellular matrix overlay comprises at least one extracellular matrix protein or fragment thereof. Extracellular matrix proteins for use as an overlay include, but are not limited to, collagen I, collagen III, collagen IV, fibronectin, laminin, vitronectin, gelatin, or combinations thereof. In some embodiments, the ECM overlay is Geltrex® matrix (Life Technologies Corporation) or Matrigel™ matrix (BD Biosciences). Geltrex® matrix and Matrigel™ matrix are composed in part of collagen IV, entactin, and laminin. The ECM overlay can be made from purified individual ECM components, from basement membrane extracted from tumor cells, or from ECM isolated from tissue, such as, for example, extracted ECM from liver. Extracellular matrix proteins, ECM components, and ECM matrices are commercially available.

ECM protein concentration suitable for use in the overlay can vary depending, for example, on the type of ECM used. In some embodiments, the ECM protein concentration can range from about 0.050 mg/ml to about 1.0 mg/ml. In certain embodiments, the ECM protein concentration ranges from 0.1 mg/ml to 0.5 mg/ml.

The ECM is generally diluted in culture media appropriate for the cells being cultured. Typically, the progenitor cells are plated and induced to differentiate prior to the application of the ECM overlay. Generally, renewal of the ECM overlay coincides with media changes and typically occurs every 2-7 days.

Agents with the capability of inducing hepatic progenitor cells or iPSCs to differentiate into hepatocyte-like cells are known in the art including, but not limited to, proteins, peptides, and small molecules. For example, DMSO, epidermal growth factor (EGF), or a combination of DMSO and EGF added to the media can induce differentiation of HepaRG™ cells (Parent et al. (2004) Gastroenterology 126:1147-1156). Hepatocyte growth factor (HGF) is also of use in differentiating progenitor cells into hepatocyte-like cells. Culturing a confluent HepaRG™ cell population with growth media containing about 2% DMSO will induce differentiation of the HepaRG™ cells primarily into hepatocyte-like and cholangiocyte-like cells. Alternatively, culturing in growth media containing about 20 ng/ml EGF will induce a HepaRG™ cell population to differentiate into hepatocyte-like and cholangiocyte-like cells.

As used herein, “hepatocyte-like cells” resemble primary human hepatocytes morphologically and/or in hepatocyte-specific marker expression. For example, hepatocyte-like cells may be organized into well-delineated trabeculae resembling those formed in primary human hepatocyte culture and resulting in the formation of a polarized epithelium and functional bile canaliculi-like structures. Using phase-contrast microscopy, for example, hepatocyte-like cells may be observed within the differentiated hepatic progenitor culture as small polarized cells with refractile borders, dark cytosol, and/or bile canaliculi. Alternatively, or in addition to the resemblance of morphological features, hepatocyte-like cells may exhibit liver-specific markers similar to primary human hepatocytes. Such expressed markers include, but are not limited to, albumin, key drug metabolizing enzymes (e.g., cytochrome P450 enzymes such as CYP3A4), signal transduction pathway components (e.g., nuclear receptors), and drug transporters (e.g., MRP2). See, for example, (Gripon et al. (2002) Proc. Natl. Acad. Sci. USA 99:15655-15660; Parent et al. (2004) Gastroenterology 126:1147-1156.

As used herein, “cholangiocyte-like cells” do not resemble primary human hepatocytes morphologically and/or in hepatocyte-specific marker expression. By phase-contrast microscopy, for example, cholangiocyte-like cells within a differentiated hepatic progenitor cell culture may appear flat with less visible nuclei, clear cytosol, and/or no refractile border. Alternatively, or in addition to the resemblance of morphological features, cholangiocyte-like cells may exhibit biliary-specific markers including, but not limited to, alpha-6 integrin and cytokeratin 19. See, for example, Gripon et al. (2002) Proc. Natl. Acad. Sci. USA 99:15655-15660; Parent et al. (2004) Gastroenterology 126:1147-1156.

In another aspect, provided are compositions comprising a cell culture comprising a population of differentiated hepatic progenitor cells overlayered with an extracellular matrix. In one such embodiment, the cell culture comprises differentiated HepaRG™ cells with an extracellular matrix overlay, for example a Geltrex®-based matrix overlay. In some embodiments, the cell culture of the compositions comprise a population of differentiated hepatic progenitor cells wherein >50% of the cells in the differentiated progenitor cell population are hepatocyte-like cells. In some embodiments, >60% of the cells in the differentiated progenitor cell population are hepatocyte-like cells. In still other embodiments, >70% of the cells, >80% of the cells, and >90% of the cells in the differentiated progenitor cell population are hepatocyte-like cells.

In one embodiment, a composition comprises a cell culture comprising a population of differentiated HepaRG™ cells with an extracellular matrix overlay, wherein >50% of the cells of the population are hepatocyte-like cells. In still other embodiments, >60% of the cells, >70% of the cells, >80% of the cells, and >90% of the cells in the differentiated HepaRG™ cell population are hepatocyte-like cells. In a particular embodiment, a composition comprises a cell culture comprising a population of differentiated HepaRG™ cells with a Geltrex®-based matrix overlay, wherein >70% of the cells of the population are hepatocyte-like cells.

In another embodiment, provided is an in vitro cell culture composition comprising a population of differentiated cells derived from a hepatic progenitor cell line, wherein the population of differentiated cells comprises >60% hepatocyte-like cells. In still other embodiments, the population of differentiated progenitor cells comprises >70%, >80%, and >90% hepatocyte-like cells. In certain embodiments, an in vitro cell culture composition comprises a population of differentiated cells derived from an iPSC, wherein the population of differentiated cells comprises >60% hepatocyte-like cells. In other embodiments, the population of differentiated iPSCs comprises >70%, >80%, and >90% hepatocyte-like cells. In a particular embodiment, a composition comprises a cell culture comprising a population of differentiated HepaRG™ cells, wherein the population of differentiated HepaRG™ cells comprises >70% hepatocyte-like cells.

Also provided are compositions prepared by the use of the disclosed methods.

Cell cultures provided herein are substantially composed of a single cell population, that of a hepatocyte-like cell that phenotypically resembles a human hepatocyte. Such cell cultures produced through the use of the overlay have improved metabolic activity compared to the cultures without the overlay. Accordingly, the differentiated cell cultures provided herein are suitable for use in any application in which primary hepatocytes are utilized. For example, the differentiated cell cultures particularly useful for the evaluation of drug clearance, drug-drug interaction, drug stability, and drug safety, including, but not limited to, metabolism, transport, and induction assays. Drug-drug interaction studies include, for example, induction and inhibition (direct and time-dependent) studies. Drug stability studies include, for example, drug stability and intrinsic clearance studies. Drug transport studies include, for example, drug uptake and drug efflux studies. Assays using hepatocytes for such studies and evaluations are well known in the art. The methods and compositions disclosed herein provide a reproducible, convenient source of hepatocyte-like cells that can replace primary human hepatocytes, for example, in such assays and evaluations.

In one embodiment, a method is provided of investigating in vitro drug metabolism is described, the method comprising incubating a hepatic progenitor cell-derived population of hepatocyte-like cells in the presence of a xenobiotic, and determining the metabolic fate of the xenobiotic, or the affect of the xenobiotic on the hepatocyte-like cells or on an enzyme or metabolic activity thereof. The population of cells in the method is at least >60% hepatocyte-like cells. In some embodiments, the method is performed with a population of differentiated HepaRG™ cells comprising >70% hepatocyte-like cells.

In embodiments of assays which measure a metabolic activity, the metabolic activity is selected from the group consisting of coumarin 7-hydroxylase (COUM), dextromethorphan O-demethylase (DEX), 7-ethoxycourmarin O-deethylase (ECOD), activities responsible for the phase II metabolism of 7-hydroxycoumarin (7-HCG and 7-HCS), mephenytoin 4-hydroxylase (MEPH), testosterone 6 (beta)-hydroxylase (TEST), tolbutamide 4-hydroxylase (TOLB), phenacetin O-deethylase (PHEN), chloroxazone 6-hydroxylase (CZX), paclitaxel hydroxylase, and bupropion hydroxylase.

Differentiated HepaRG™ cell populations prepared with an extracellular matrix overlay respond to prototypical cytochrome P450 inducers such as omeprazole (OMP), phenobarbital (PB), and rifampicin (RIF). Also, hepatocyte-like cells from differentiated HepaRG™ cells support constitutive androstane receptor (CAR) translocation. Treatment of the cells with indirect CAR activators results in CAR accumulates in the nucleus of the cells. Accordingly, the cell populations have utility in the evaluation of in vitro enzyme induction and receptor translocation.

Metabolic stability studies are typically performed to estimate a drug candidate's metabolic half-life and intrinsic clearance rates. The hepatocyte-like cells derived from differentiated HepaRG™ cells cultured under an extracellular matrix overlay have expression levels of key metabolic enzymes and nuclear receptors consistent with levels observed in primary human hepatocytes. Accordingly, the hepatocyte-like cell populations are suitable for assessing the metabolic stability of candidate drug compounds.

The HepaRG™ cell-derived hepatocyte-like cells are also tolerant of long culture periods (e.g., >14 days, >21 days) and so are well-suited for in vitro determination of acute and chronic toxicity resulting, for example, from intrinsic and/or metabolism-based mechanisms.

The following examples are provided by way of illustration and not by way of limitation.

### EXAMPLES

### Example 1

Hepatic progenitor cells were expanded and differentiated as follows. Undifferentiated HepaRG™ cells were seeded in a culture vessel at about 27,000 cells/cm² in HepaRG™ cell growth medium (Williams E Medium, 1× GlutaMax, 1× Pen/Strep, 800 ug/mL insulin, 25 mg/mL hemisuccinate hydrocortisone, 10% fetal bovine serum (FBS)) and cultured at 37° C. with 5% CO₂ for about 14 days. During this culture period, media changes occurred every 2-3 days and the cell number expanded, reaching 100% confluency between 10-14 days.

After 14 days in HepaRG™ cell growth media, the cells were overlayed with Geltrex® matrix, an extracellular matrix, and cultured in HepaRG™ differentiation media (Williams E Medium, 1× GlutaMax™, 1× Pen/Strep, 800 ug/mL insulin, 25 mg/mL hemisuccinate hydrocortisone, 10% FBS, 2% DMSO). The cells were maintained in these conditions for 14 days and differentiation media was renewed every 2-3 days and the overlay was renewed every 2-7 days. Other HepaRG™ cells were overlayed with Geltrex® matrix and treated in the same way but with the serum-free differentiation media. For a control, a parallel culture of HepaRG™ cells were differentiated without a Geltrex® matrix overlay.

By phase-contrast microscopy, hepatocyte-like clusters were observed within the differentiated HepaRG™ culture composed of small polarized cells with refractile borders, dark cytosol, and bile canaliculi. The cells resembled primary human hepatocytes morphologically. The hepatocyte-like cells were organized into well delineated trabeculae closely resembling those formed in primary human hepatocyte culture resulting in the formation of a polarized epithelium and functional bile canaliculi-like structures. The hepatocyte-like cells also exhibited liver-specific markers similar to primary human hepatocytes including expression of albumin, key drug metabolizing enzymes (i.e., CYP3A4), signal transduction pathways (i.e., nuclear receptors), and drug transporters (i.e., MRP2).

By phase-contrast microscopy, cholangiocyte-like cells within the differentiated HepaRG™ culture appeared flat with less visible nuclei, clear cytosol, and no refractile border. These cells did not resemble primary human hepatocytes morphologically or exhibit liver-specific markers (e.g., CYP3A4, albumin) but were positive for the biliary cell marker alpha-6 integrin.

Cryopreserved HepaRG™ cells (Life Technologies Corporation) are terminally differentiated prior to cryopreservation. Frozen HepaRG™ cells were thawed and plated per manufacturer's instructions and placed into 37° C., 5% CO₂ cell culture incubator. 24 hours after plating, media was aspirated and cells were overlayed using 0.35 mg/mL Geltrex® matrix in 4° C. in plating medium (herein referred to as “770 medium”, Life Technologies Corp. Cat. No. HPRG770), serum containing HepaRG™ Induction medium (referred to herein as “740” medium, Life Technologies Corp. Cat. No. HPRG740), or serum-free HepaRG™ Induction medium (referred to herein as “750” medium, Life Technologies Corp. Cat. No. HPRG750). Control cells were also cultured in parallel without an overlay in the 770, 740, or 750 media. The overlay was renewed every 2-7 days and the media renewed every 2-3 days.

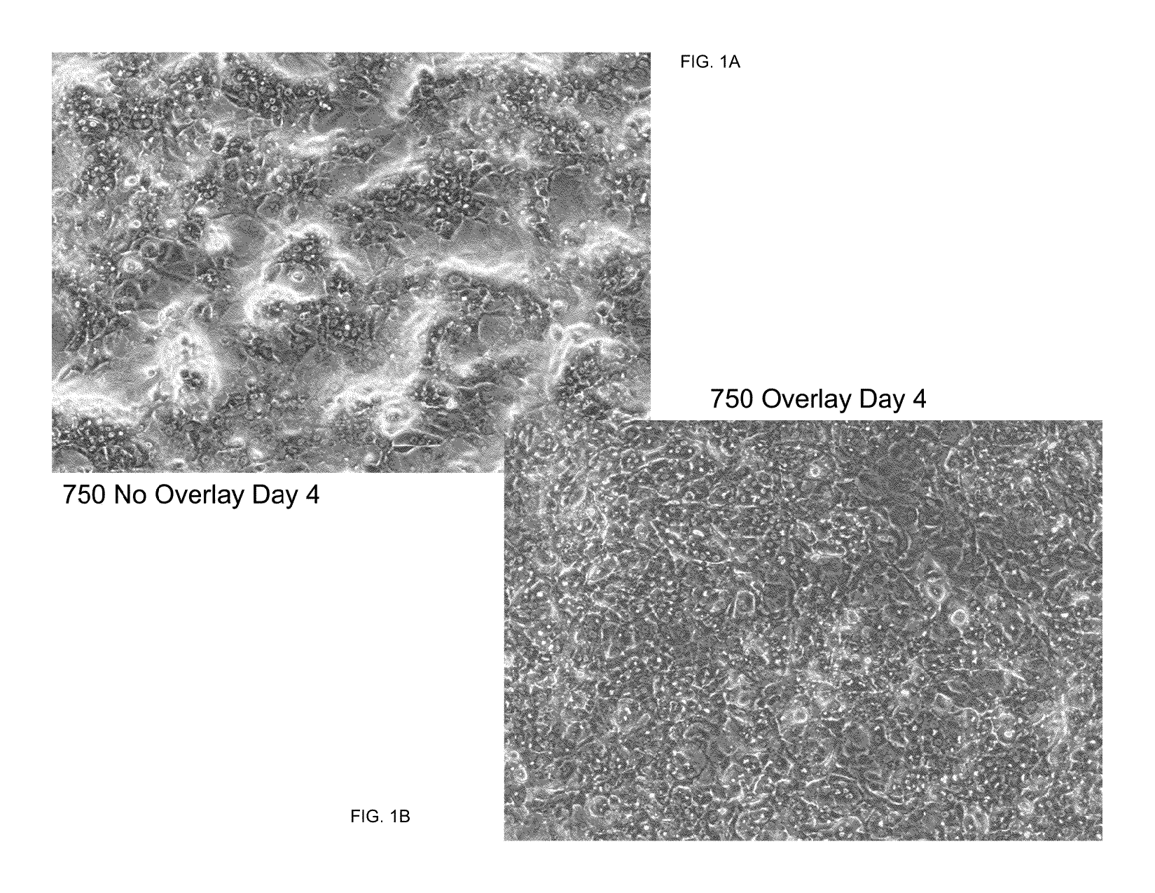

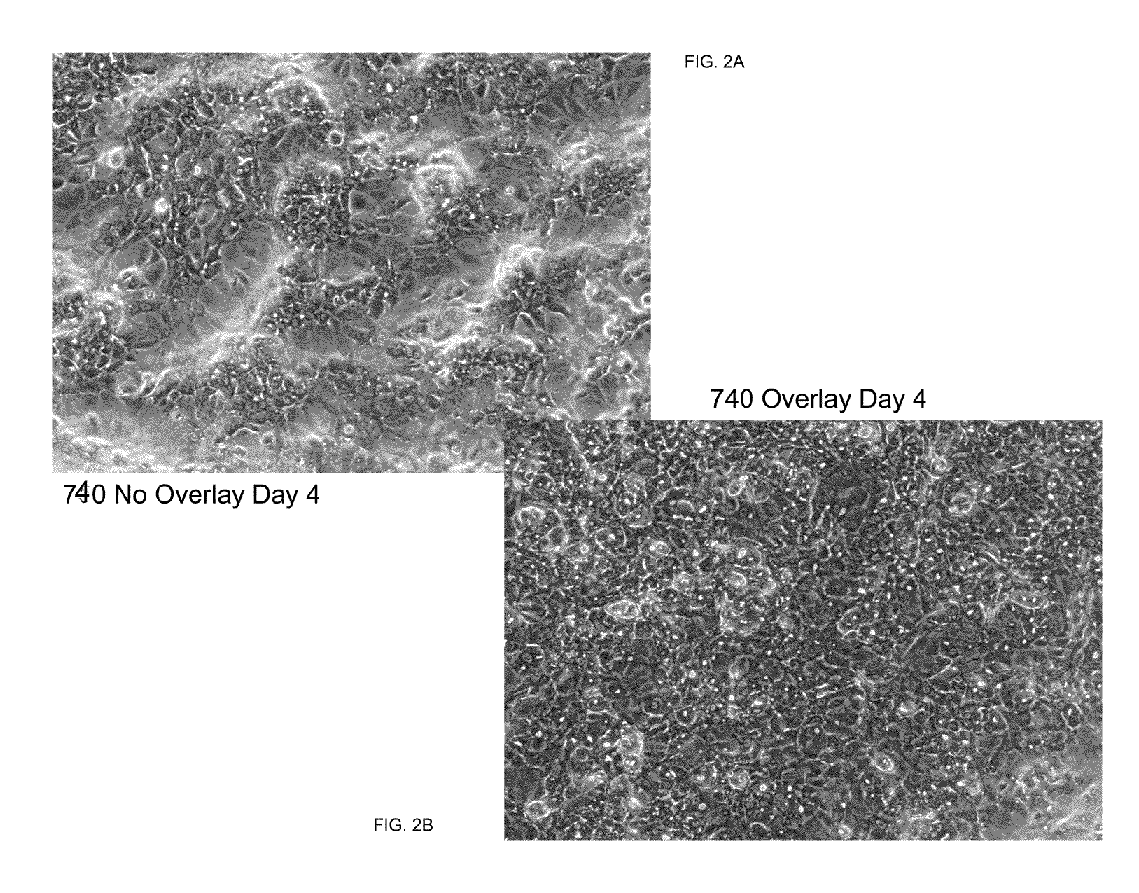

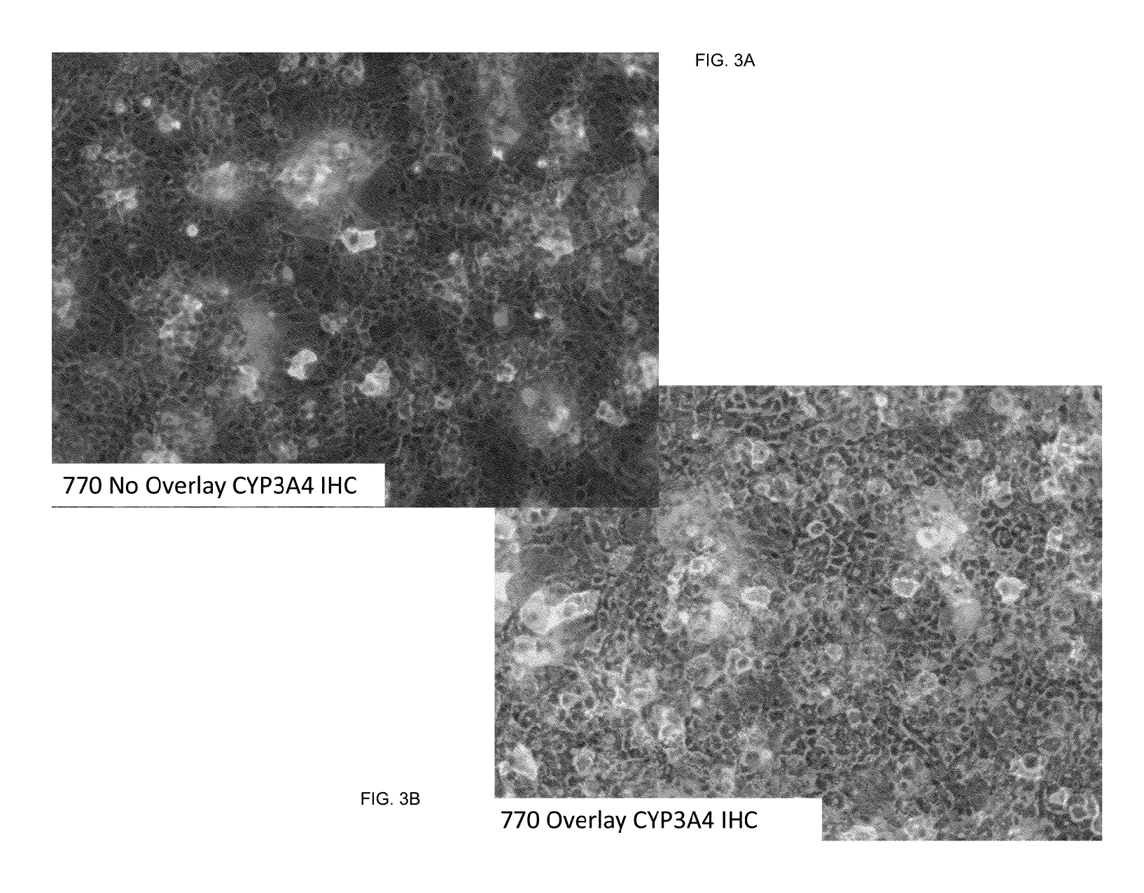

Exemplary phase-contrast microscopy images of the cryopreserved differentiated HepaRG™ cells are shown in FIGS. 1, 2, and 3. The cells of FIGS. 1A and 1B were cultured in serum-free medium and the cells of FIGS. 2A and 2B were cultured in medium containing serum. The cells of FIGS. 3A and 3B were cultured in 770 plating medium without and with an overlay, respectively. Anti-CYP3A4 immunohistochemistry analysis of the cells in FIGS. 3A and 3B indicates the increase in CYP3A4 expression in the cell population cultured with the overlay.

When cultured with the extracellular matrix overlay, HepaRG™ cells preferentially differentiated into hepatocyte-like cells and the resultant cell culture was substantially composed of hepatocyte-like cells (FIG. 1B, FIG. 2B, and FIG. 3B). The cultures with an overlay also had a reduction in the number of cholangiocyte-like cells compared to cultures without an overlay. Without the overlay, HepaRG differentiation led to a resultant cell culture of about 50% cholangiocyte-like cells and about 50% hepatocyte-like cells (FIG. 1A, FIG. 2A, and FIG. 3A).

### Example 2

HepaRG™ cells respond to prototypical cytochrome P450 inducers and are useful in the evaluation of in vitro enzyme induction. The P450 induction screening was evaluated with HepaRG cell populations cultured with and without an extracellular matrix overlay.

Cryopreserved HepaRG™ cells (Life Technologies Corporation) are terminally differentiated prior to cryopreservation. Frozen HepaRG™ cells were thawed and plated per manufacturer's instructions and placed into 37° C., 5% CO₂ cell culture incubator. 24 hours after plating, media was aspirated and cells were overlayed using 0.35 mg/mL Geltrex® matrix in 4° C. in 770 media. 72 hours after plating, media was aspirated and media was exchanged with either 740 or 750 media containing 0.1% DMSO, 50 μM omeprazole (OMP), 1 mM Phenobarbital (PB), or 10 μM Rifampicin (RIF). 740 and 750 media containing vehicle or prototypical inducers was replaced daily for a total of 72 hours of treatment. Day 6 of culture (24 hours after last dosing), P450 activity was evaluated and total RNA was isolated. P450 activity was evaluated utilizing in situ incubations of prototypical substrates (100 μM phenacetin, 500 bupropion, 10 μM midazolam, or 200 μM testosterone) in Hanks' Balanced Salts Solution (HBSS) for 1 hour at 37° C. shaking on microplate shaker @600 rpm. After in situ incubations were completed, incubation matrix (supernatant/HBSS) was removed from cell monolayer and metabolite formation was evaluated by LC/MS-MS analysis. Microphotographs were taken daily to monitor morphological changes.

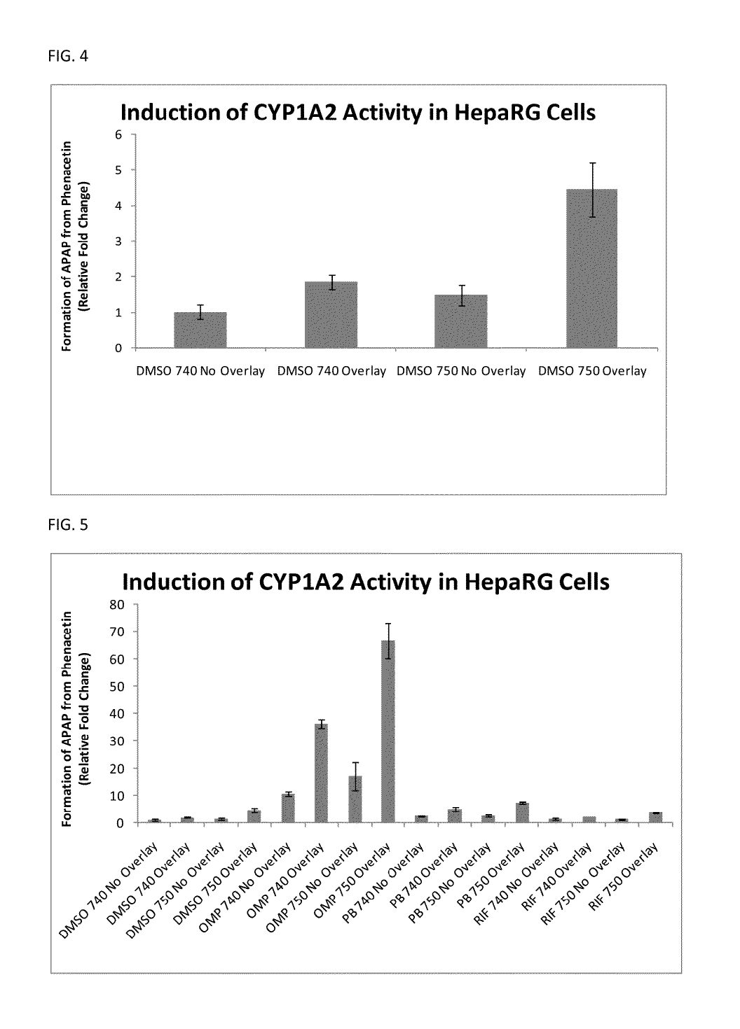

Results of such induction assays are shown in FIGS. 4-15 for cells cultured in HepaRG™ Induction medium (740, containing serum) and in serum-free HepaRG™ Induction medium (750) and for cells treated with an overlay and without an overlay.

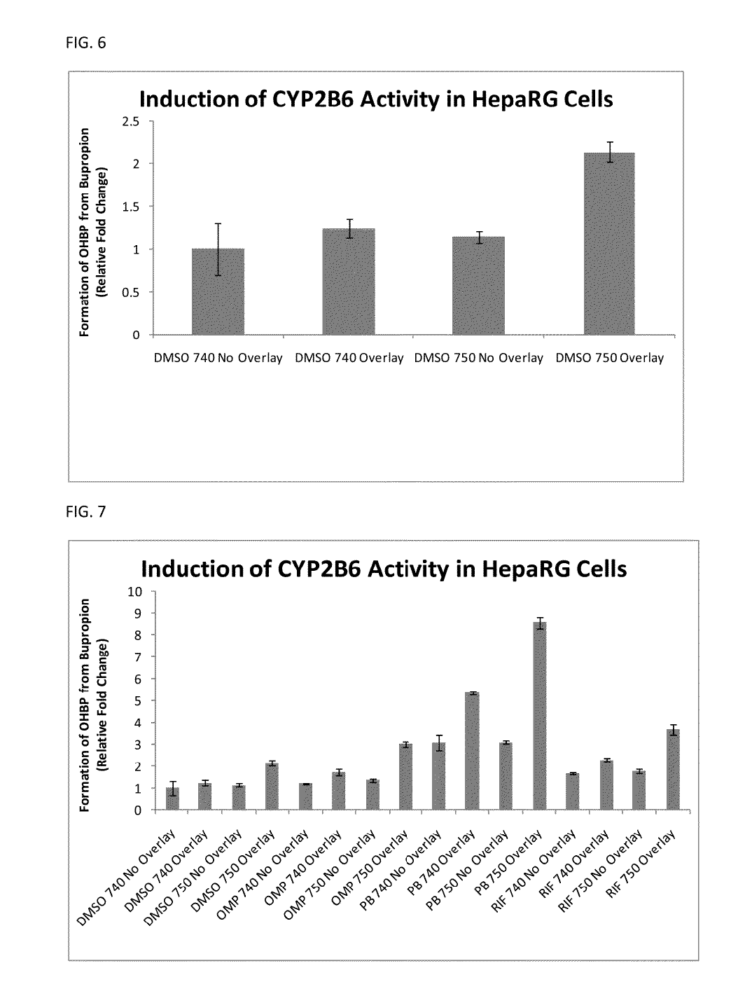

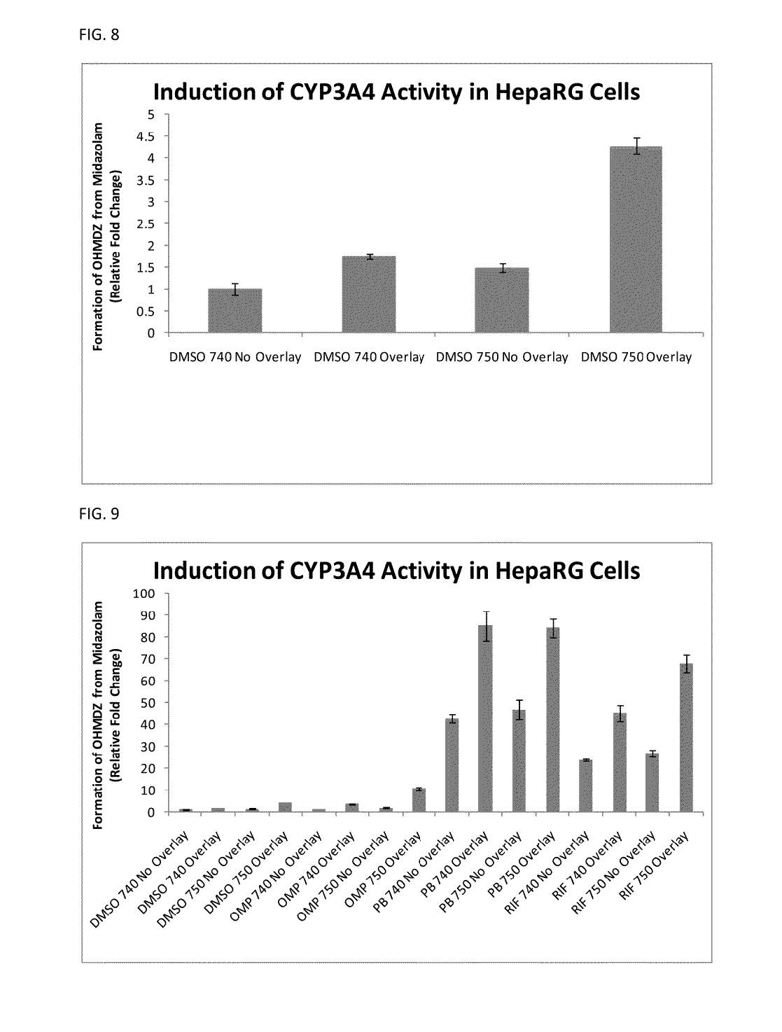

FIGS. 4 and 5 depict induction of CYP1A2 activity in HepaRG™ cells as the relative fold change in formation of acetaminophen (APAP) from phenacetin in response to DMSO, OMP, PB, and RIF. FIGS. 6 and 7 depict induction of CYP2B6 activity in HepaRG™ cells as the relative fold change in formation of hydroxybupropion (OHBP) from bupropion in response to DMSO, OMP, PB, and RIF. FIGS. 8 and 9 depict induction of CYP3A4 activity in HepaRG™ cells as the relative fold change in formation of 1-hydroxymidazolam (OHMDZ) from midazolam in response to DMSO, OMP, PB, and RIF. Baseline P450 enzyme activities increased in the presence of the overlay. When the overlay was used in conjunction with serum-free media, a synergistic increase in P450 enzyme activity over baseline was observed.

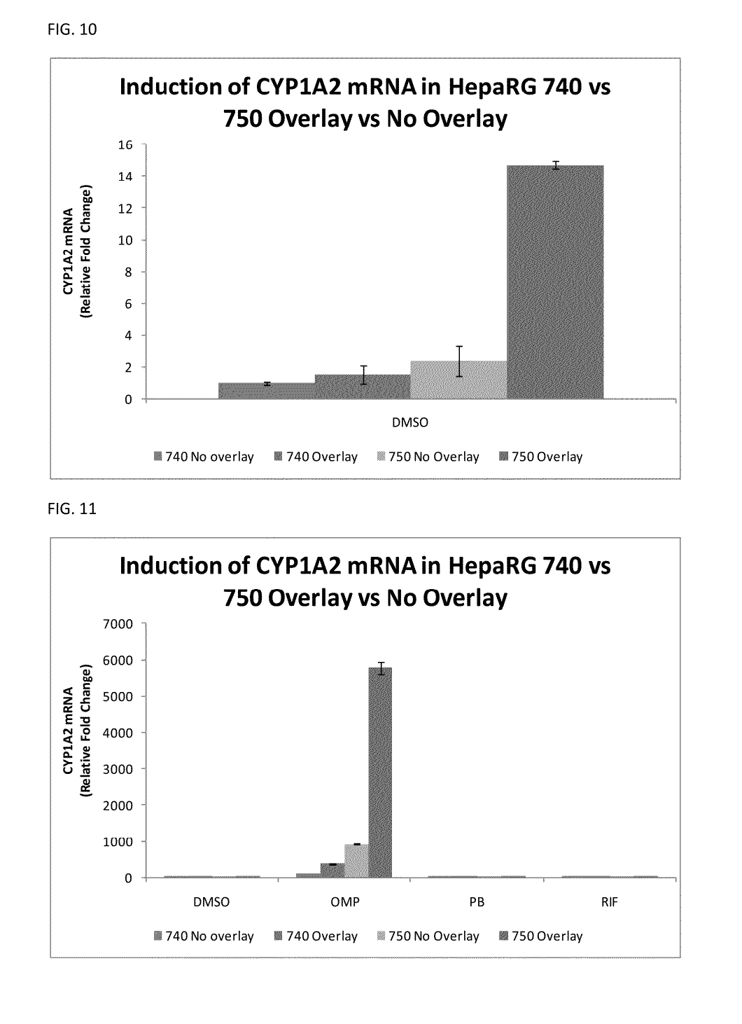

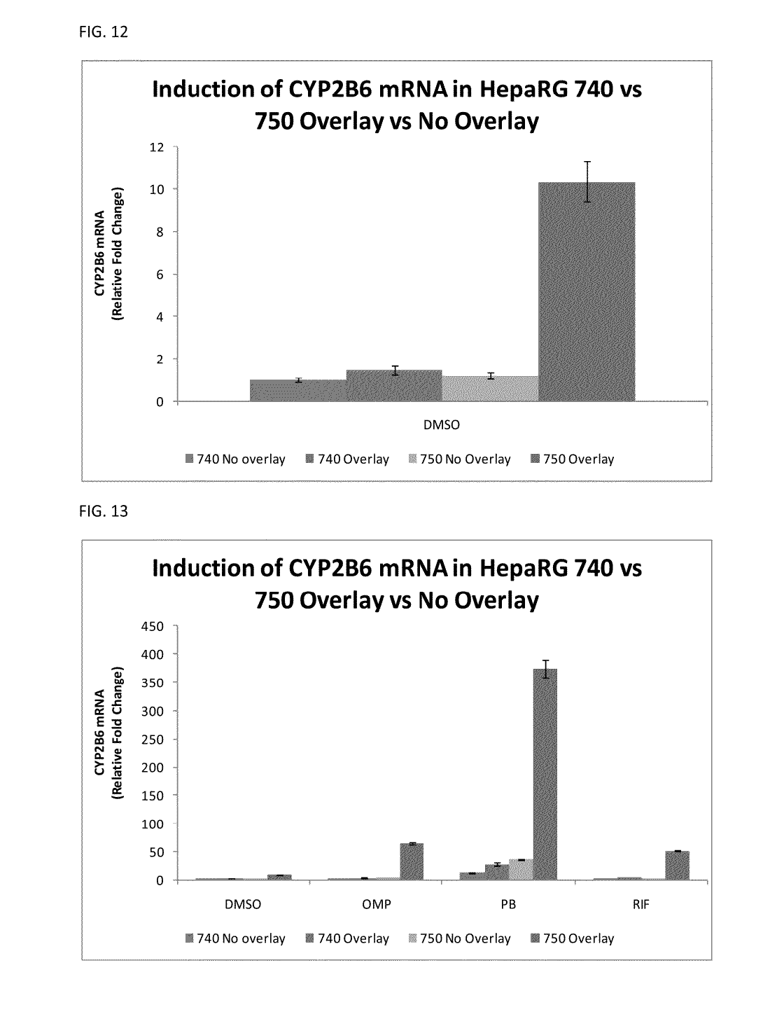

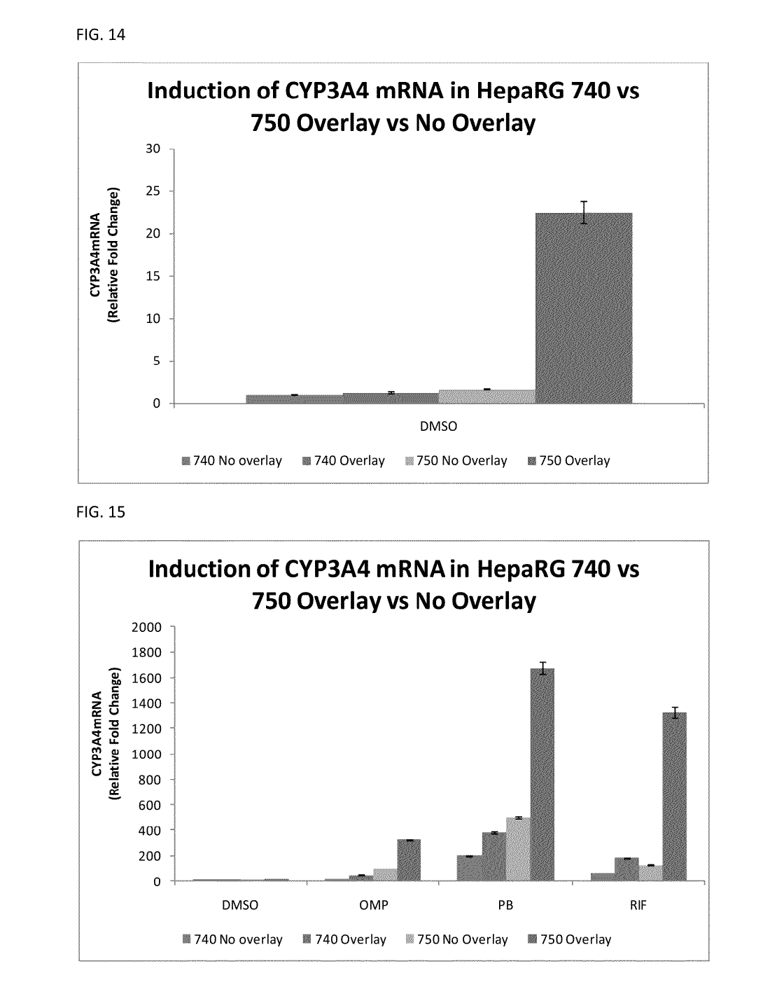

This P450 enzyme activity data is supported by P450 enzyme mRNA data. FIGS. 10 and 11 depict induction of CYP1A2 mRNA in HepaRG™ cells as the relative fold change in CYP1A2 mRNA in response to DMSO, OMP, PB, and RIF. FIGS. 12 and 13 depict induction of CYP2B6 mRNA in HepaRG™ cells as the relative fold change in CYP2B6 mRNA in response to DMSO, OMP, PB, and RIF. FIGS. 14 and 15 depict induction of CYP3A4 mRNA in HepaRG™ cells as the relative fold change in CYP3A4 mRNA in response to DMSO, OMP, PB, and RIF.

As can be seen with these results, use of the extracellular matrix overlay increases the response of the target gene regulated by xenobiotic activated receptors (constitutive androstane receptor (CAR), pregnane X receptor (PXR), aryl hydrocarbon receptor (AhR)) in the presence of prototypical P450 inducers. Typically, RIF induces CYP2B6 enzyme activity in primary human hepatocytes via PXR activation, however, RIF does not induce CYP2B6 in HepaRG™ cells unless the cells are overlayed. Also, the induction response was better when serum was removed from the culture medium. Culturing with a matrix overlay resulted in the signal transduction signal in HepaRG™ cells to become more similar to that observed in primary human hepatocytes than culturing without the overlay.

### Example 3

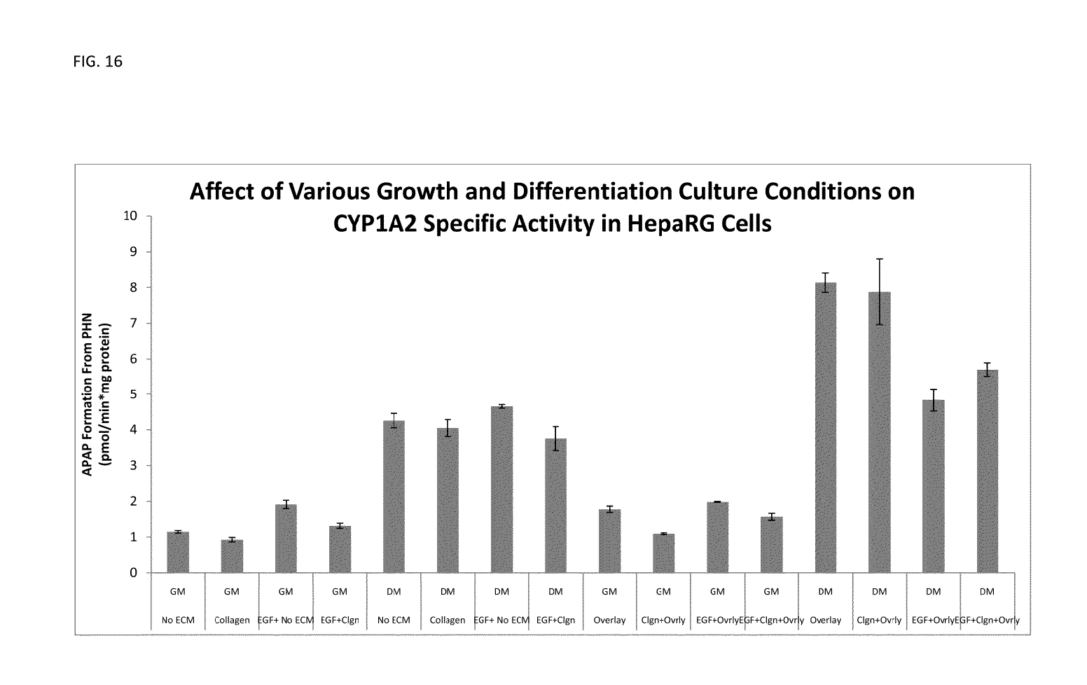

An extracellular matrix overlay was used during HepaRG™ progenitor cell differentiation and resulted in a cell culture substantially composed of hepatocyte-like cells. Undifferentiated HepaRG™ cells were plated in a culture vessel, allowed to attach to the vessel, and overlayed with Geltrex® matrix in Differentiation media. Some cells were cultured with the overlay, others on a collagen coated plate, others in the presence of 20 mg/ml EGF, and combinations thereof (overlay+collagen, overlay+EGF, collagen+EGF, overlay+EGF+collagen). For a control, a parallel sets of HepaRG™ cells were cultured in growth medium instead of differentiation medium. In situ P450 specific activity assays were performed using cells from the various cultures and the results are depicted in FIGS. 16-18.

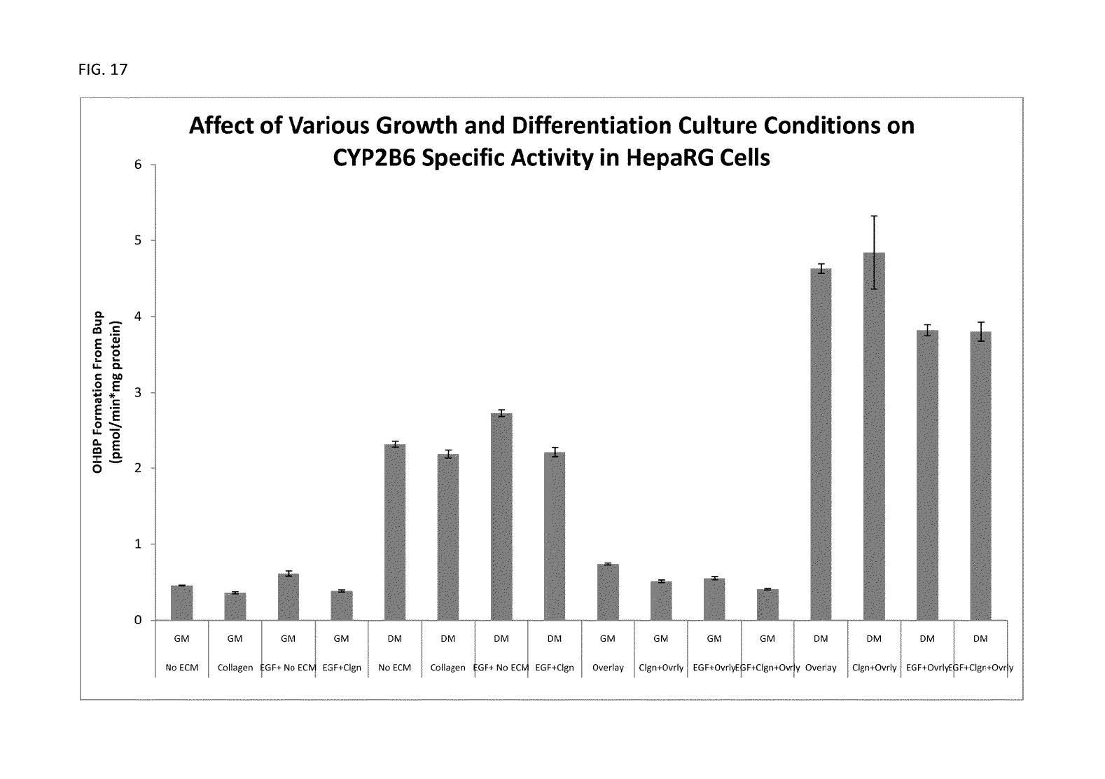

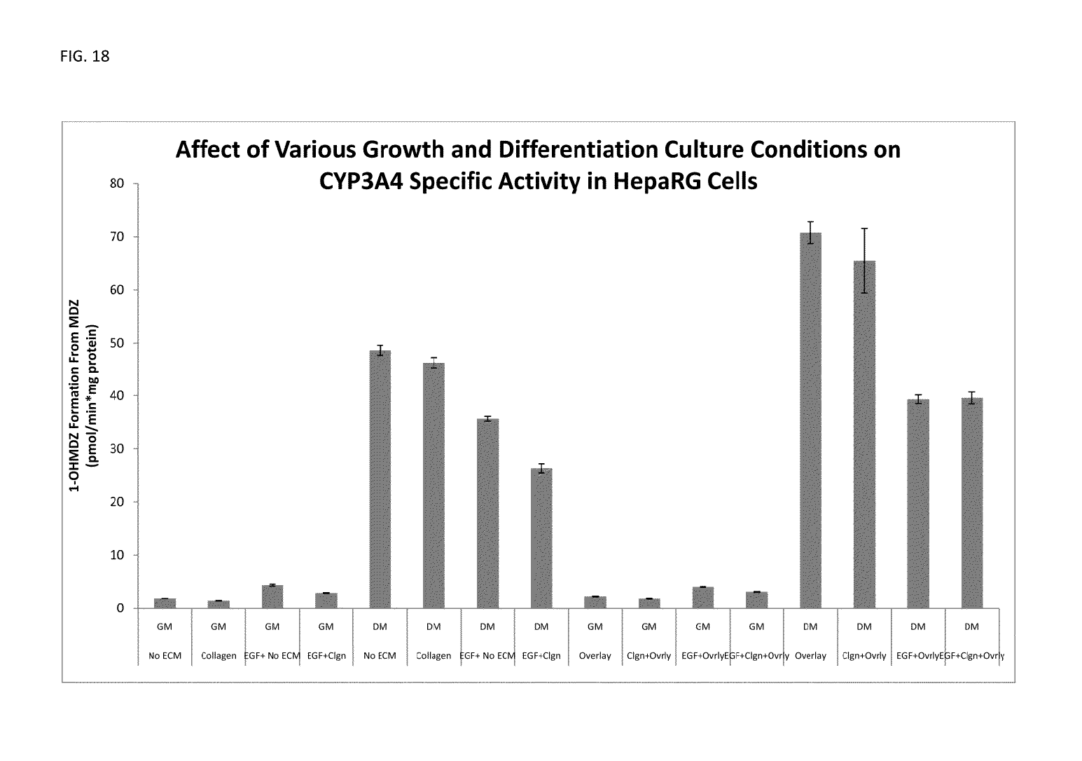

FIG. 16. depicts CYP1A2 activity in the cell cultures as the change in formation of acetaminophen (APAP) from phenacetin. FIG. 17 depicts CYP2B6 activity in the cell cultures as the change in formation of hydroxybupropion (OHBP) from bupropion. FIG. 18 depicts CYP3A4 activity in the cell cultures as the change in formation of 1-hydroxymidazolam (OHMDZ) from midazolam. P450 enzyme activities were very low in cells cultured only in growth media (GM) but clearly measurable in cells cultured in differentiation media (DM) indicating differentiation into a population of hepatocyte-like cells. The differentiated cells cultured with the overlay resulted in a significant increase in P450 enzyme activities compared to that of the differentiated cells cultured without the overlay. In some conditions, the overlay resulted in cells having double the P450 enzyme activity as would be expected if the percentage of hepatocyte-like cells doubled in the overlay culture.

Use of the extracellular matrix overlay during HepaRG™ progenitor cell differentiation resulted in a single population of hepatocyte-like cells and thus a population with improved hepatocyte-like function (e.g., increased P450 activity).

What is claimed is:

1. A method for making a preparation substantially comprised of hepatocyte-like cells, the method comprising:
inducing differentiation of hepatic progenitor cells and culturing the cells with an overlay comprising at least one extracellular matrix protein or fragment thereof.

2. The method of claim 1, wherein the hepatic progenitor cells are induced to differentiate before culturing with the overlay.

3. The method of claim 1, wherein the hepatic progenitor cells are induced to differentiate while culturing with the overlay.

4. The method of claim 1, wherein the hepatic progenitor cells are HepaRG™ cells.

5. The method of claim 1, wherein the preparation comprises >70% hepatocyte-like cells

6. An in vitro cell culture comprising a population of differentiated cells derived from a hepatic progenitor cell line, wherein population of differentiated cells comprises >70% hepatocyte-like cells.

7. The in vitro cell culture of claim 6, wherein the population of differentiated cells comprises >80% hepatocyte-like cells.

8. The in vitro cell culture of claim 6, wherein the hepatic progenitor cell line is HepaRG™ cell line.

9. The in vitro cell culture of claim 8, population of differentiated cells comprises >90% hepatocyte-like cells.

10. A composition comprising an in vitro population of differentiated cells derived from a hepatic progenitor cell line, and a matrix overlay comprising at least one extracellular matrix protein or fragment thereof.

11. The composition of claim 10, wherein the matrix overlay comprises at least three extracellular matrix proteins or fragments thereof.

12. The composition of claim 10, further comprising an agent capable of inducing differentiation of the progenitor cell line.

13. The composition of claim 10, wherein the population of differentiated cells comprises >70% hepatocyte-like cells.

14. The composition of claim 13, wherein the population of differentiated cells comprises >80% hepatocyte-like cells.

15. The composition of claim 10, wherein the hepatic progenitor cell line is HepaRG™ cell line.

16. A differentiated population of HepaRG™ cells comprising >70% hepatocyte-like cells.

17. The differentiated population of claim 16, wherein the population comprises >80% hepatocyte-like cells.

18. The differentiated population of claim 17, wherein the population comprises >90% hepatocyte-like cells.

19. A method for increasing cytochrome P450 activity in an in vitro cell preparation, the method comprising:
inducing differentiation of hepatic progenitor cells,
culturing the cells with a matrix overlay for at least one day, the overlay comprising at least one extracellular matrix protein or fragment thereof, and
measuring cytochrome P450 activity in the cell preparation, whereby the activity in the cell preparation is increased relative to a culture of the progenitor cells differentiated and cultured without a matrix overlay.
